(19) United States
(12) Patent Application Publication
(10) Pub. No.: US 2022/0280440 A1
(43) Pub. Date: Sep. 8, 2022

(54) METHOD FOR THE MANUFACTURE OF A DOSAGE FORM WITH MUCOADHESIVE PROPERTIES FOR BUCCAL ADMINISTRATION OF BIOLOGICS

(71) Applicant: UNIVERSIDAD DE CHILE, Santiago (CL)

(72) Inventors: Javier MORALES, Santiago (CL); Miguel MONTENEGRO, Santiago (CL); Felipe CAMPANO, Santiago (CL)

(21) Appl. No.: 17/751,419

(22) Filed: May 23, 2022

Related U.S. Application Data
(63) Continuation-in-part of application No. 16/070,138, filed on Jul. 13, 2018, now abandoned.

(30) Foreign Application Priority Data
Jan. 15, 2016 (CL) 112-2016

(51) Int. Cl. A61K 9/70; A61K 9/00; A61K 38/19; A61K 38/21; A61K 38/28; A61K 38/26; A61K 38/37; A61K 39/29; A61K 38/08; A61K 38/47; A61K 38/45; A61K 47/36; A61K 47/10; A61K 47/28; A61K 47/32; A61K 47/38

(52) U.S. Cl. CPC A61K 9/7007; A61K 9/006; A61K 38/19; A61K 38/212; A61K 38/215; A61K 38/217; A61K 38/28; A61K 38/191; A61K 38/26; A61K 38/37; A61K 39/29; A61K 38/085; A61K 38/47; A61K 38/45; A61K 47/36; A61K 47/10; A61K 47/28; A61K 47/32; A61K 47/38

(57) ABSTRACT

The invention relates to the pharmaceutical industry, particularly to the pharmaceutical industry related to biological drugs, biomacromolecules, biopharmaceuticals, or biologics. More particularly, the invention relates to a method for the manufacture of a highly controlled and stable dosage form with mucoadhesive properties for buccal administration of biologics such as lymphokines, hormones, hematopoietic factors, growth factors, antibodies, enzymes, inhibitors, vaccines, DNA, and RNA derivatives, among others. Based on inkjet printing, the invention uses inks comprising the biologic(s) included in a nanosystem, particularly in polymeric nanoparticles.

### CROSS-REFERENCE TO RELATED APPLICATIONS

This patent application is a Continuation-In-Part of U.S. patent application Ser. No. 16/070,138 filed on Jul. 13, 2018, which is a National Stage application of International Application No. PCT/CL2017/050001 filed on Jan. 6, 2017, which claims foreign priority benefit of Chilean Patent Application No. 0112-2016 filed on Jan. 15, 2016, the entire contents of all the above applications are incorporated herein by reference.

### TECHNICAL FIELD OF THE INVENTION

The present invention relates to the pharmaceutical industry, particularly the pharmaceutical industry related to the administration of biological products (biologics). In particular, the present invention relates to a method for the manufacture of a highly controlled and stable dosage form with mucoadhesive properties for buccal administration of biologics such as lymphokines, hormones, hematopoietic factors, growth factors, antibodies, enzymes, inhibitors, vaccines, DNA, and RNA derivatives, among others.

### BACKGROUND OF THE INVENTION

Drugs can be administered by several routes, being the oral route one of the most convenient, safest, and economical alternative for a patient. However, this route has disadvantages since some drugs are poorly or erratically absorbed in the gastrointestinal tract or are destroyed by gastric acids. For example, biologic products (biologics, also called biomacromolecules) such as insulin, antibodies, DNA or RNA derivatives, proteins, or enzymes are almost entirely formulated and administered for injectable routes (intravenous, subcutaneous, or intramuscular) due to their sensitiveness to the gastrointestinal tract conditions: acidic pH in the stomach, proteolytic enzymes along the gastrointestinal tract, the presence of bile salts, and microbiota in the large intestine. Thus, these molecules are prone to be degraded when administered by the oral route.

On the other hand, the injectable route also has disadvantages, wherein the pharmacokinetics of the injected biologic is one of the main problems. For example, these molecules may have reduced circulating half-life, limiting their use. Furthermore, injected biologic may have an indiscriminate distribution, resulting in undesired side effects. Other known limitations of this route are associated with pain management, usually leading the patients to delay their therapies. For example, in the treatment of diabetes, this phenomenon is known as psychological insulin resistance.

Given all the limitations of injectable and oral routes, pharmaceutical development has been focused on finding alternative routes for the administration of biologics. However, research has been done for years without success.

The search for alternative routes has not been easy. Only two insulins administered by alternative routes have been approved by the FDA: the now retired Exubera (Mack, G. S. (2007). Pfizer dumps Exubera. Nat Biotech, 25(12), 1331-1332), and the currently marketed Afrezza® (MannKind Corporation). Both technologies have been developed for administration by the pulmonary route.

Another alternative route of administration is the buccal route. The buccal administration of insulin, lysozyme, and albumin has been proposed, as disclosed in WO2014065870A1; Morales, J. O., et al. (2013). Protein-coated nanoparticles embedded in films as delivery platforms. J Pharm Pharmacol, 65(6), 827-838; Morales, J. O. et al. (2014). Films loaded with insulin-coated nanoparticles (ICNP) as potential platforms for peptide buccal delivery. Colloids Surf B Biointerfaces, 122, 38-45; Giovino, C., et al. (2012). Development and characterisation of chitosan films impregnated with insulin loaded PEG-b-PLA nanoparticles (NPs): a potential approach for buccal delivery of macromolecules. Int J Pharm, 428(1-2), 143-151. These documents describe polymeric films incorporating nanoparticles loaded with peptides and proteins during the manufacturing process and casting. Specifically, the documents disclose mucoadhesive films with embedded particles comprising biologics for the buccal delivery of said biologics. These particles are incorporated into the film during the manufacturing process; thus, the biologic is in contact with solvents and excipients, and it is exposed to the polymeric film drying conditions.

Other alternative administration routes, such as nasal, pulmonary, ocular, vaginal, and rectal, among others, do not use mucoadhesive polymer films. The skin, which could use films, does not allow the passage of biologics except for physical skin modifications (for example, generating pores).

Studies for administering biologics through the buccal route have been focused on films obtained by different methods or through sprays containing nanodroplets for absorption. For example, polymeric films consisting of a solid solution or dispersion of chitosan and ethylenediaminetetraacetic acid containing insulin have been described (Cui F., et al. (2009). Preparation and evaluation of chitosan-ethylenediaminetetraacetic acid hydrogel films for the mucoadhesive transbuccal delivery of insulin. J Biomed Mater Res A, 89A(4), 1063-1071). However, that system does not contemplate the incorporation of insulin in nanoparticles. Additional alternatives for the buccal administration of insulin have been developed, such as an insulin spray (Modi P., et al. (2002). The evolving role of oral insulin in the treatment of diabetes using a novel RapidMist System. Diabetes Metab Res Rev, 18 (51): S38-42; Guevara-Aguirre J., et al. (2004). Beneficial effects of addition of oral insulin spray (Oralin) on insulin secretion and metabolic control in subjects with type 2 diabetes mellitus suboptimally controlled on oral hypoglycemic agents. Diabetes Technol Ther, 6(1): 1-8; Sadrzadeh N., et al. (2007). Peptide drug delivery strategies for the treatment of diabetes. J Pharm Sci, 96(8): 1925-1954).

The technology of printing biologics has been explored in the last few years, but it is still poorly described in the literature. This technology has been used for immobilizing biomolecules on surfaces for biosensors, analytical techniques, or controlled delivery of protein-based drugs. For example, Ihalainen P., et al. disclose a piezoelectric and thermal printing strategy but using the biomolecules without being encapsulated with nanosystems (Ihalainen P., et al. (2015). Printing technologies for biomolecule and cell-based applications. Int J Pharm, 494(2), 585-592). The same occurs in the printing technology used for fabricating DNA microarrays, in which the DNA solutions are deposited onto glass substrates without using nanosystems (Okamoto T., et al. (2000). Microarray fabrication with covalent attachment of DNA using Bubble Jet technology. Nat Biotechnol, 18, 438-441). Hence, the biomolecules are exposed to heat and ink solvents during the printing process. Other research has been focused on demonstrating that the printing of bare biologics maintains its structure at least partially (Zheng Q., et al. (2011). Application of inkjet printing technique for biological material delivery and antimicrobial assays. Anal Biochem, 410(2): 171-6). All these investigations have omitted the use of nanosuspensions as strategies to formulate biologics.

Further attempts related to the technology of printing biologics are disclosed in several patent documents. For example, WO2014144512 (Aprecia Pharmaceuticals Company) teaches a 3D printed dosage form of rapid dispersion and high doses of levetiracetam (anticonvulsant) and a process for preparing the said dosage form. The levetiracetam is in a porous matrix that disperses in water in less than 10 seconds. WO2014188079 (Abo Akademi University) relates to oral dosage forms of vitamin(s) and/or dietary mineral(s) or nicotine produced by printing techniques and a method for producing the same. TWI364442 (Du Pont) teaches a method for depositing a printable inkjet composition to a substrate comprising: depositing an ink composition on a substrate by inkjet printing; wherein said composition comprises: (a) functional material; (B) organic polymer comprising polyvinyl pyrrolidone; dispersed in (c) dispersion vehicle selected from organic solvent, water or mixtures thereof; and wherein the viscosity of the said composition is between 5 mPa·s-50 mPa·s at a temperature of 25 to 35° C. Moreover, this document teaches inkjet printing at least one patterned layer of the above composition in a substrate for fabricating an electronic component.

None of the above patent documents disclose methods for the manufacture of dosage forms based on inkjet printing of inks containing nanosystems loaded with drugs.

In consequence, there is a need in the prior art for obtaining new pharmaceutical dosage forms for the administration of biologics in a highly stable and controlled way using an alternative route different than the injectable and oral route and methods for obtaining said new pharmaceutical dosage forms without losing the activity of the biologics.

### BRIEF DESCRIPTION OF THE INVENTION

The present invention relates to a method for manufacturing a dosage form with mucoadhesive properties for the buccal administration of biologics or biomacromolecules, wherein the method is based on inkjet printing technology. The manufacturing method of the present invention provides a highly predictable and reproducible way to deposit a biologic onto the substrate with mucoadhesive properties, and it is uniformly distributed over the same. As a result, the dosage form obtained by this process has an even dose of the biologic, ensuring an accurate and efficient administration of said biologic to the patient.

One of the main advantages of this manufacturing method is the use of nanosystems, which are essential to obtain a dosage form with a precise amount of active biologic. Thus, the nanosystems provide the biologic protection from the harsh conditions of the inkjet printing process, such as heat and other ink components that may cause destabilization of the molecule. Furthermore, the nanosystems significantly contribute to controlling the biologic release, stability, and drug delivery.

In addition, the method for manufacturing the dosage form provides the biologic to be released in the buccal cavity and permeates through the buccal mucosa in a highly stable and controlled manner.

The method for manufacturing the dosage form of the present invention comprises the steps of a) preparing polymeric nanoparticles including at least one biologic, wherein the polymeric nanoparticles are prepared by mixing a polymer with cationic monomeric units and a polymer with anionic monomeric units in a charge ratio (n⁺/n⁻) of 0.1 to 1; b) preparing a printing ink with a suspension of the polymeric nanoparticles including the at least one biologic; and c) printing onto a polymeric film with said printing ink.

The biologic is preferably selected from the group consisting of a lymphokine, a hormone, hematopoietic factors, growth factors, antibodies, enzymes, inhibitors, and vaccines; wherein said lymphokine is selected from aldesleukin cytokine, antineoplastic protein denileukin diftitox, recombinant interleukin Oprelvekin, interferon-α1, interferon α2a, interferon-α2b, interferon β1a, interferon γ1b, interferon γ1b, and tumoral necrosis factor human-α1a (TNFα-1a) tasonermin; wherein said hormone is selected from human insulin, insulin lispro, insulin aspart, insulin glulisine, insulin glargine, insulin detemir, glucagon, somatropin, somatrem, follitropin-α, follitripin-β, choriogonadotropin-α, lutropin-α, calcitonin, teriparatide, preotact, thyrotropin-α, nesiritide, and angiotensin 1-9; wherein said hematopoietic factors are selected from filgrastim, lenogastrim, sargramostim, molgramostim, epoetin-α, epoetin-β, epoetin-γ, and darbepoetin-α; wherein said growth factors are selected from mecasermin, rinfabate mecasermin, nepidermin, becaplermin, palifermin, dibotermin-α, and eptotermin-α; wherein said antibodies are selected from Fab fragments such as arcitumomab, digoxin Fab, abciximab, certolizumab; murine antibodies such as muramonab-CD3, capromab, ibritumomab tiuxetan, toistumomab; chimeric antibodies such as rituximab, infliximab, basiliximab, cetuximab, brentuximab vedotin; humanized antibodies such as daclizumab, trastuzumab, palivizumab, gemtuzumab ozogamicin, alemtuzumab, efalizumab, omalizumab, bevacizumab, natalizumab, ranibizumab, eculizumab, tocilizumab; human antibodies such as adalimumab, panitumumab, golimumab, canakinumab, ustekinumab, ofatumumab, denosumab, belimumab, and ipilimumab; wherein said enzymes are selected from imiglucerase, agalsidase-β, alglucosidase-α, laronidase, idursulfase, galsulfase, factor VIIa, factor VIII, factor IX, drotrecogin-α, alteplase, reteplase, tenecteplase, dornase-α, rasburicase, lysozyme, and ribonuclease; wherein said inhibitors are selected from desirudin, lepirudin, antithrombin III, ecallantide, and anakinra; and wherein said vaccines are selected from human hepatitis vaccine and human papilloma virus vaccine.

In a preferred embodiment, the biologic is selected from the group consisting of lysozyme, ribonuclease, angiotensin 1-9, and insulin.

In a preferred embodiment, the polymer with cationic monomeric units is a polymethacrylate derivative with ionizable tertiary amine groups in its monomeric units, and the polymer with anionic monomeric units is alginate.

In a preferred embodiment for preparing the printing ink, a viscosity agent is added to the suspension of the polymeric nanoparticles, said viscosity agent is selected from the group consisting of glycerol and propylene glycol. Preferably, the viscosity agent is glycerol.

In another preferred embodiment for preparing the printing ink, an absorption enhancing agent may be added to the suspension of the polymeric nanoparticles. Such absorption enhancing agent may be selected from deoxycholic acid, taurocholic acid, glycodeoxycholic acid, glycocholic acid, and taurodeoxycholic acid.

In a preferred embodiment, the method of the present invention further comprises preparing the polymeric film with a polymer selected from the group consisting of cellulose, cellulose derivatives, polyvinyl pyrrolidone, polyvinyl alcohol, chitosan, alginate, agar, carrageenan, guar gum, xanthan gum, polycarbophil, polyacrylic acid derivatives, poly(methacrylic acid) derivatives, and combinations thereof. Preferably, the poly(methacrylic acid) derivative is a cationic polymethacrylate, and the cellulose derivative is selected from the group consisting of hydroxypropyl cellulose, hydroxyethyl cellulose, and carboxymethyl cellulose.

In a preferred embodiment, an aqueous solution of any of the above-mentioned polymers in a concentration of 10% w/v is used for preparing the polymeric film.

In a preferred embodiment, a plasticizing agent may be added to the aqueous solution of the polymer for obtaining a ratio range of plasticizing agent:polymer of 1:9 to 3:7 in the polymeric film. Preferably, the plasticizing agent is glycerol.

### BRIEF DESCRIPTION OF THE FIGURES

FIG. 1 is a diagram showing the manufacture of polymeric films loaded with biologics. Films are used as printing substrates, and the ink is formulated as a suspension of the nanosystem to allow proper dosing of the biologic. The dosage form obtained by the printing process of the present invention is placed and adheres to the buccal epithelium due to its mucoadhesive properties.

FIGS. 2A, 2B, and 2C show the particle size (nm), polydispersity index, and zeta potential (mV), respectively, of polymeric nanoparticles loaded with lysozyme, before (pre-print) and after (post-print) being printed on the inert surface. Different polymer charge ratios (n+/n−) were analyzed.

FIGS. 3A and 3B show graphs of the amount of lysozyme that remains on the film after the printing process versus the surface area of the film printed. Inks with different lysozyme concentrations (0.15, 0.5, 3.5, and 10 mg/mL) and different printing surface areas (4, 9, 16, and 49 cm²) were used for both pure lysozyme (FIG. 3A) and as a mixture with sodium deoxycholate (DCH) (FIG. 3B).

FIGS. 4A, 4B, and 4C show the results of the percentage of the relative activity of lysozyme (Lys) (FIGS. 4A and 4B) and RNase (FIG. 4C) with and without the addition of the absorption enhancing agent deoxycholate (DCH) at different concentrations of lysozyme (0.15, 0.5, 3.5, and 10 mg/mL) and RNase (10 mg/mL) in different printing surface areas (4, 9, 16, and 49 cm²), after the printing process of the ink on the printing surfaces.

FIG. 5 shows an image obtained by scanning electron microscope (SEM) of a sample of a nanoemulsion with lysozymes. The bar represents 500 nm.

FIGS. 6A and 6B show the association efficiency (%) of nanoemulsions (NE) with lysozymes as a function of Miglyol/Lecithin ratio after the printing process and the effect of the said process on the physicochemical characteristics (average size (nm) and polydispersity index (PDI)) of a subsequent nanoemulsion print in different printing surface areas.

FIG. 7A shows the effect of printing ink suspensions of polymeric nanoparticles with or without lysozymes on the physicochemical characteristics (average size (nm) and polydispersity index (PDI)) of the nanoparticles before (pre-print) and after (post-print) the printing process. FIG. 7B shows a graph with the percentage of lysozymes released (Lyz) in time from different polymeric nanoparticle formulations. The Eudragit® E and alginate nanoparticles with different charge ratios (CR: 0.5, 1.33, 0.5, and 1.33) and different total charges (TC: 30, 30, 20, 20) were tested. The total charges (TC) are the number of positive and negative charges that each polymer forming the nanoparticle provides. FIG. 7C shows an image of the polymeric nanoparticles obtained by SEM (bar represents 500 nm).

FIGS. 8A and 8B show the particle size (nm), polydispersity index (PDI), and Zeta potential (mV) of nanoparticles before (pre-print) and after (post-print) printing inks suspensions containing polymeric nanoparticles loaded with insulin, with a polymer charge ratio of 0.5 (negative) and 2 (positive). FIG. 8C shows the percentage of association efficiency of insulin after printing inks containing polymeric nanoparticles loaded with insulin, with a polymer charge ratio of 0.5 (negative) and 2 (positive).

FIG. 9A shows a graph of the effects of the printing process on the mucoadhesive properties (maximum adhesive force (mN)) of different polymeric films (ethylcellulose (Ethocel™), cross-linked polyacrylic acid polymer (Carbopol®), HPMC K100 10 g, HPMC K100 20 g, HPMC K100 30 g, HPMC K100 10 g (1 print), HPMC K100 10 g (3 prints), HPMC K100 10 g (7 prints)). FIGS. 9B and 9C show the tensile strength (N/mm²) and elastic modulus (N/mm²/%)), respectively, of polymeric films of different grades of HPMC (HPMC K15M 10 g, HPMC K15M 20 g, HPMC K15M 30 g, HPMC K15M 10 g (1 print), HPMC K15M 20 g (1 print), HPMC K15M 30 g (1 print)).

FIGS. 10A, 10B, and 10C show the effects of charge ratio (n⁺/n⁻) and the total sum of charges (n⁺+n⁻) on the average size (FIG. 10A), zeta potential (FIG. 10B), and polydispersity index (FIG. 10C) of polymeric nanoparticles. FIGS. 10D and 10E show SEM images of the polymeric nanoparticles with a total sum of charges (n⁺+n⁻) equal to 6 μM.

FIG. 11A shows the effect of different core precursors for obtaining nanoparticles by antisolvent co-precipitation on the average size and polydispersity index. FIG. 11B shows an SEM image of D,L-valine nanoparticles obtained by antisolvent co-precipitation process (scale 500 nm).

FIGS. 12A and 12B show the amount of RNase-A deposited on the film after the ink printing process as a function of the surface area of the film printed. Inks with different RNase-A concentrations (0.15, 0.5, 3.5, and 10 mg/mL) and different printing areas (4, 9, 16, and 49 cm²) were used for both pure RNase-A (FIG. 12A) and as a mixture with sodium deoxycholate (DCH) (FIG. 12B).

FIG. 13. shows FTIR spectra of lysozyme with glycerol (above) and lysozyme only (below) as model ink for the printing process.

### DETAILED DESCRIPTION OF THE INVENTION

The present invention is a method for manufacturing a highly controlled and stable dosage form with mucoadhesive properties for the buccal administration of biologics. This method uses printing inks comprising nanosystems with an active ingredient such as a biologic. More specifically, the nanosystem used is a polymeric nanoparticle, which is prepared by mixing a polymer with cationic monomeric units (polycation) and a polymer with anionic monomeric units (polyanion), more particularly, in a charge ratio polycation/polyanion (n⁺/n⁻) of 0.1 to 1. The nanosystem comprises at least one biologic selected from lymphokines, hormones, hematopoietic factors, growth factors, antibodies, enzymes, inhibitors, vaccines, and DNA or RNA derivatives. Preferably, the biologic may be selected from lysozyme, ribonuclease, angiotensin 1-9, and insulin. The nanosystem may comprise one or more biologics.

It is important to highlight that the dosage form for buccal administration of the present invention should not be understood as equivalent to a dosage form for oral administration. In the case of oral administration, the entire dosage form is swallowed. The dosage form of the present invention has been designed to have mucoadhesive properties; thus, it can be placed or attached to the buccal cavity, specifically to the buccal mucosa.

The following description of the invention and the accompanying figures are provided for the purposes of illustration and a comprehensive understanding of various embodiments of the present invention as defined by the claims. It is not intended to be exhaustive or limit the invention to the precise forms disclosed, and it is to be understood that all equivalents and/or combinations of embodiments are intended to be included in this description. It will be apparent to those of ordinary skill in the art that various changes and modifications may be made to the described embodiments without altering the scope and spirit of the present invention. In addition, descriptions of well-known functions and constructions may be omitted for clarity and conciseness.

All technical and scientific terms used to describe the present invention have the same meaning understood by a person having basic knowledge in this technical field. Notwithstanding, to define the scope of the invention more clearly, a list of terminology used in this description is included herewith.

As used herein, the term “biologic” is intended to describe biological molecules having pharmacological activity, such as peptides, proteins, growth factors, plasmids, nucleic acids including DNA, RNA, antisense oligonucleotides, and attenuated vaccines, among others. It should also be understood as similar terms “biomacromolecules”, “biological drugs”, “biomolecules”, and “biopharmaceutical drugs”.

In a preferred embodiment of the present invention, the biologics may be selected from those disclosed in Ryu J. K., et al. (2012). Current status and perspectives of biopharmaceutical drugs. Biotechnol Bioproc E, 17(5):900-11, which is incorporated by reference herein in its entirety. Preferably, the biologics are selected from lymphokines, hormones, hematopoietic factors, growth factors, antibodies, enzymes, inhibitors, and vaccines.

In particular, the biologic is selected from the group consisting of a lymphokine, a hormone, hematopoietic factors, growth factors, antibodies, enzymes, inhibitors, and vaccines; wherein said lymphokine is selected from aldesleukin cytokine, antineoplastic protein denileukin diftitox, recombinant interleukin Oprelvekin, interferon α1, interferon α2a, interferon-α2b, interferon β1a, interferon β1b, interferon γ1b, and tumoral necrosis factor human-α1a (TNFα-1a) tasonermin; wherein said hormone is selected from human insulin, insulin lispro, insulin aspart, insulin glulisine, insulin glargine, insulin detemir, glucagon, somatropin, somatrem, follitropin-α, follitripin-β, choriogonadotropin-α, lutropin-α, calcitonin, teriparatide, preotact, thyrotropin-α, nesiritide, and angiotensin 1-9; wherein said hematopoietic factors are selected from filgrastim, lenogastrim, sargramostim, molgramostim, epoetin-α, epoetin-β, epoetin-γ, and darbepoetin-α; wherein said growth factors are selected from mecasermin, rinfabate mecasermin, nepidermin, becaplermin, palifermin, dibotermin-α, and eptotermin-α; wherein said antibodies are selected from Fab fragments such as arcitumomab, digoxin Fab, abciximab, certolizumab; murine antibodies such as muramonab-CD3, capromab, ibritumomab tiuxetan, tositumomab; chimeric antibodies such as rituximab, infliximab, basiliximab, cetuximab, brentuximab vedotin; humanized antibodies such as daclizumab, trastuzumab, palivizumab, gemtuzumab ozogamicin, alemtuzumab, efalizumab, omalizumab, bevacizumab, natalizumab, ranibizumab, eculizumab, tocilizumab; human antibodies such as adalimumab, panitumumab, golimumab, canakinumab, ustekinumab, ofatumumab, denosumab, belimumab, and ipilimumab; wherein said enzymes are selected from imiglucerase, agalsidase-β, alglucosidase-α, laronidase, idursulfase, galsulfase, factor VIIa, factor VIII, factor IX, drotrecogin-α, alteplase, reteplase, tenecteplase, dornase-α, rasburicase, lysozyme, and ribonuclease; wherein said inhibitors are selected from desirudin, lepirudin, antithrombin III, ecallantide, and anakinra; and wherein said vaccines are selected from human hepatitis vaccine and human papilloma virus vaccine.

As used herein, the term “nanosystem” or “nanocarrier” refers to a particle useful for drug delivery that has any shape and at least one dimension in the nanometer scale, usually with one or more dimensions less than 500 nm, preferably all the dimensions less than 500 nm. The particles may be spherical, spheroid-like, or irregular. It could be polymer or polymeric nanoparticles, protein-coated nanoparticles, nanospheres, nanocapsules, liposomes, micelles, nanoemulsions, or any other nanosystem known in the state of the art. In particular, all the dimensions of the polymeric nanoparticles described herein are less than 500 nm, more preferably, less than 300 nm.

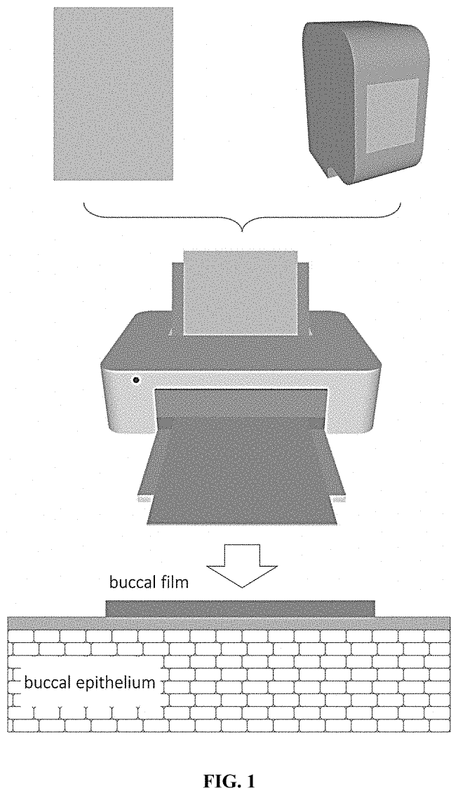

The method of the present invention for the manufacture of a dosage form with mucoadhesive properties for buccal administration of a biologic comprises the steps of a) preparing a nanosystem with a biologic; b) preparing a suspension of the nanosystem with the biologic for obtaining an ink, and d) printing on the surface of the polymer film with the ink. A brief illustration of the method is shown in FIG. 1.

In a preferred embodiment of the present invention, preparing a nanosystem with a biologic comprises selecting a nanosystem suitable for the biologic. Among the nanosystem alternatives (polymer nanoparticles, protein-coated nanoparticles, nanocapsules, or nanoemulsions), the polymer nanoparticles (also called polymer nanoparticles, nano complexes, or polyelectrolyte complexes) showed to be the most suitable nanocarriers for the biologics. Even more, the polymeric nanoparticles prepared using the complex coacervation technique had the best results. This technique involves the electrostatic interaction of a polycation (a polymer with cationic monomer units) with a polyanion (a polymer with anionic monomeric units). Interestingly, after studying the effect of the charge ratio (positive charges/negative charges: n⁺/n⁻) in the formation of polymeric nanoparticles, it was observed that the best polymeric nanoparticles obtained, in terms of size homogeneity and stability, were obtained in the presence of an excess of poly anion.

In a preferred embodiment, the polymer with cationic monomeric units (polycation) used in the present method is a polymethacrylate derivative with ionizable tertiary amine groups in its monomeric units, and the polymer with anionic monomeric units (polyanion) used in the present method is alginate.

Different techniques may be used to prepare the other alternative nanosystems with the biologic. For example, nanoemulsions and nanocapsules may be prepared by solvent displacement method, wherein an organic phase containing oil such as medium-chain triglycerides (e.g., Miglyol), a surfactant selected from lecithin, cetylpyridinium chloride, and hexadecyltrimethylammonium bromide, and a water-miscible organic solvent selected from acetone and ethanol, are incorporated into an aqueous solution to form the nanoemulsions. Protein-coated nanoparticles may be prepared by antisolvent co-precipitation method. The preparation of the protein-coated nanoparticles may further comprise the addition of a saccharide selected from lactose, mannitol, and sorbitol.

After preparing the nanosystem with the biologic, particularly the polymeric nanoparticles with the biologic prepared by the complex coacervation technique, a suspension of the polymeric nanoparticles is prepared for obtaining the printing ink. The preparation of the said suspension (also called nanosuspension) may include a step of adding a viscosity agent to the said suspension for obtaining the printing ink. Preferably, the viscosity agent used is selected from the group consisting of glycerol and propylene glycol, but it is not limited to these alternatives. In a preferred embodiment, the preparation of the suspension includes adding glycerol as the viscosity agent.

The preparation of the printing inks may also comprise a step of adding an absorption enhancing agent to the polymeric nanoparticles' suspension, wherein the absorption enhancing agent may be selected from deoxycholic acid, taurocholic acid, glycodeoxycholic acid, glycocholic acid, and taurodeoxycholic acid, without being limited to these alternatives.

The method also comprises the step of printing onto the polymeric film with the printing ink. The polymeric film comprises film-forming polymers such as cellulose, cellulose derivatives such as hydroxypropyl cellulose, hydroxyethyl cellulose, and carboxymethyl cellulose; polyvinyl pyrrolidone, polyvinyl alcohol, chitosan, alginate, agar, carrageenan, guar gum, xanthan gum, polycarbophil, polyacrylic acid derivatives, and poly (methacrylic acid) derivatives, preferably a cationic polymethacrylate. In a more preferred embodiment of the present invention, the polymer of the polymeric film is hydroxypropylmethyl cellulose, which may be of different grades.

The polymeric film may be obtained from commercially available polymeric films or may be prepared, preferably comprising the steps described as follows. An aqueous solution of one or a mixture of the previously mentioned polymers is prepared. The solvent of the aqueous solution may be water, any other suitable solvent for the polymer, or a mixture of those solvents. In a preferred embodiment, the aqueous solution of the polymer is a water solution with the polymer, being said polymer hydroxypropylmethyl cellulose (HPMC). The said solution is prepared in a concentration of 10% w/v.

Additionally, a plasticizing agent may be added to the aqueous solution of the polymer. In a preferred embodiment, the plasticizing agent is added to the aqueous solution of the polymer in such proportion that the ratio range of plasticizing agent:polymer in the polymeric film is between 1:9 to 3:7. Preferably, the plasticizing agent is glycerol.

The preparation of the polymeric nanoparticles' suspension and the preparation of the polymeric film may also comprise each an optional step of adding one or more pharmaceutically acceptable excipients. The excipients that may be used are well known in the state of the art, such as those described in Rowe, Raymond C., Paul J. Sheskey, and Sian C. Owen. (2006). Handbook of pharmaceutical excipients. London: Pharmaceutical Press, which is incorporated by reference herein in its entirety.

Although the present invention has been optimized for its application in the pharmaceutical industry, all industries related to biologics could benefit from it. The present invention could be of interest to those companies using films as dosage forms, such as 3M healthcare, LTS Lohmann, and BDSI, among others.

The following examples are intended to illustrate the invention and its preferred embodiments, but they should not be considered under any circumstances to restrict the scope of the invention, which is determined by the content of the claims attached hereto.

### EXAMPLES

### Early Developments of the Nanosystems

The first advances of this technology were focused on studying different types of nanoparticles: polymeric nanoparticles, nanoemulsions, nanocapsules, and protein-coated nanoparticles. Due to the morphology and size achieved with the protein-coated nanoparticles obtained by antisolvent co-precipitation (Le A. D., et al. (2015). Investigation of Antisolvent Manufacturing Co-precipitation of Protein-Coated Particle (PCP) Precursors using a Variable Injection Speed Linear Actuator. In: 2015 AAPS Annual Meeting and Exposition Orlando, Fla., USA; Le A. D., et al. (2015). Formulation and Optimization of Protein-Coated Nanoparticles. In: 42nd Annual Meeting and Exposition of the Controlled Release Society Edinburgh, Scotland), it was chosen to continue the development of inks having nanosystems based on polymeric nanoparticles (also referred to as nano complexes) obtained through coacervation and nanoprecipitation and also continuing with protein-coated nanoparticles (protein-stabilized nanoparticles). The developments also continued with nanoemulsions and nanocapsules.

Due to their potential, polymeric nanoparticles were studied to deliver a peptide with cardiovascular effects (angiotensin 1-9) (Sepulveda-Rivas S., et al. (2015). Toxicity Evaluations of Polymeric Nanoparticles: Cell Viability versus Physicochemical Properties of Nanoparticles. In: 2015 AAPS Annual Meeting and Exposition Orlando, Fla., USA; Morales J. O., et al. (2015). Novel nanostructured polymeric carriers to enable drug delivery for cardiovascular diseases. Curr Pharm Des, 21(29), 4276-84), nanoparticles for delivery to the central nervous system (Catalan-Figueroa J., et al. (2015). Polymeric Nanocarriers for Drug delivery: Improving physicochemical parameters. In: NanoDDS Meeting 2015 Seattle, Wash., USA; Catalan-Figueroa J., et al. (2016). Nanomedicine and nanotoxicology: the pros and cons for neurodegeneration and brain cancer. Nanomedicine, 11(2), 171-187; Gajardo-Lopez U., et al. (2015). Nanoprecipitation of Eudragit RS and RL to Fabricate Nanostructured delivery Systems for Curcumin. In: Annual AAPS 2015 Meeting and Exposition. Orlando, Fla., USA; Jara M. O., et al. (2015). A Systematic Analysis of the Manufacture of Polymeric Nanoparticles by Nanoprecipitation (Solvent Diffusion) by Artificial Neural Networks. In: Annual AAPS 2015 Meeting and Exposition. Orlando, Fla., USA), and nanosuspensions for use as printing inks (Sepulveda-Rivas S., et al. (2015). Toxicity Evaluations of Polymeric Nanoparticles: Cell Viability versus Physicochemical Properties of Nanoparticles In: 2015 AAPS Annual Meeting and Exposition Orlando, Fla., USA; Fritz H., & Morales J. O. (2015). The Use of Eudragit E/Alginate Polymeric Nanoparticles as a Novel Drug Delivery System for Biomacromolecules. a Case Study of Lysozyme. In: 2015 AAPS Annual Meeting and Exposition Orlando, Fla., USA).

Printing Experiments with Naked Biologics

In parallel, printing experiments on inert substrates with solutions containing a biologic model (lysozyme (Lys) and ribonuclease (RNAse)) were done to evaluate the effect of the printing process on naked o pure biologics. Additionally, experiments including an absorption enhancer agent (deoxycholate (DCH) as a conventional absorption enhancer) were done to evaluate its effect on the biologic. For those purposes, aqueous solutions (Milli-Q® water) of lysozyme or RNase were prepared at 0.15, 0.5, 3.5, and 10 mg/mL. In addition, solutions with the same concentrations of lysozyme or RNase were spiked with 25% w/w DCH based on the mass of lysozyme or RNase. These aqueous solutions were mixed with glycerol as the ink viscosity agent to formulate the printing inks, in a proportion of aqueous solution/glycerol of 7:3 v/v.

The printing inks were loaded into clean cartridges compatible with HP Deskjet 1000 printer. Inert substrate surfaces of 4, 9, 16, and 49 cm² were printed with the inks. The ink printed on the surfaces was collected by refluxing with 1.0 mL of Milli-Q® water. The amount of printed lysozyme or RNase and their remaining activity were quantified.

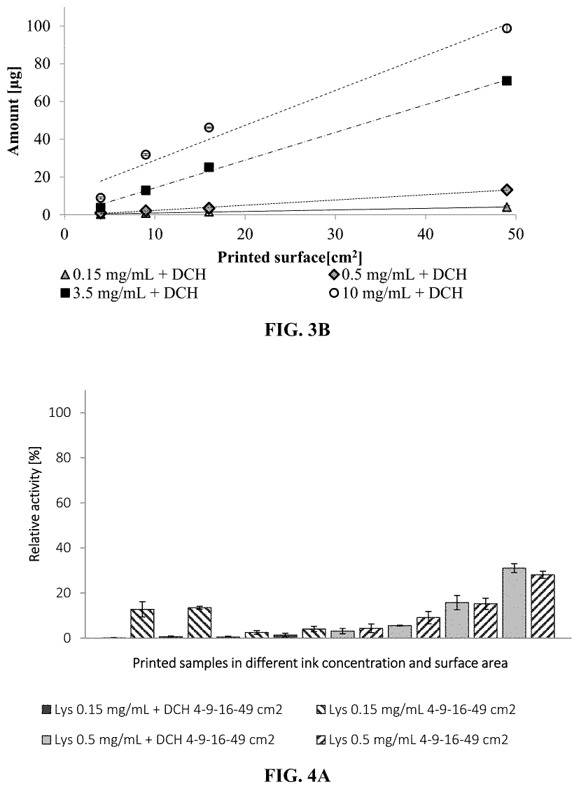

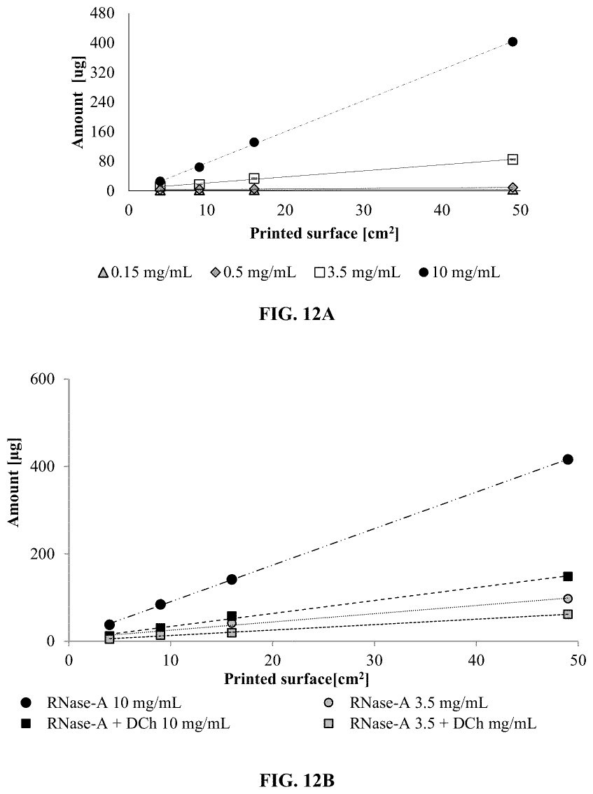

FIGS. 3A and 3B show the amount of lysozyme printed in each surface area, with or without DCH, and FIGS. 12A and 12B show the amount of RNase printed in each surface area, with or without DCH. These results demonstrated that the printing process allows obtaining controlled doses of biologics and increasing linearly with respect to the printed surface. Additionally, incorporating an absorption enhancer, such as deoxycholate, maintains the same linear trends in the printing process with lysozyme but with a decrease in the amount printed under the same surface and lysozyme concentration conditions.

Thus, the printing process was highly predictable and reproducible for naked biologics (as shown, for example, in FIGS. 3A and 3B for lysozyme solutions). Therefore, it was also expected to be reproducible for nanosystems inks.

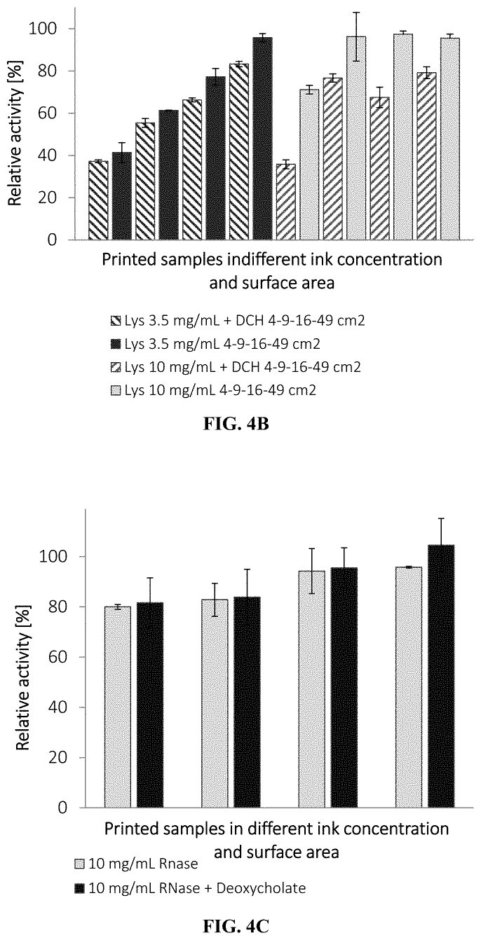

FIGS. 4A and 4B show the relative activity (%) of the printed surface area with the inks having different concentrations of lysozyme, with or without DCH, and FIG. 4C shows the relative activity (%) of the printed surface area with the inks having the same concentration of RNase, with or without DCH.

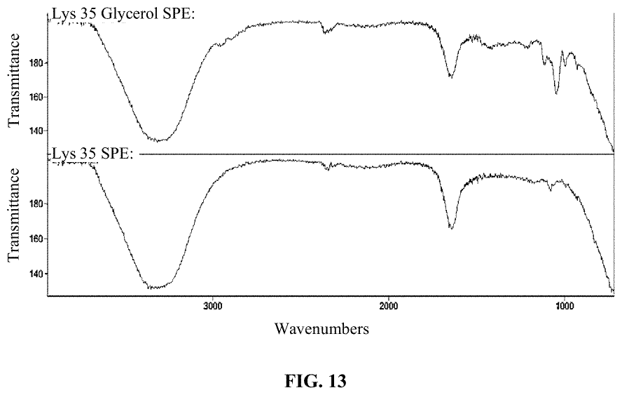

On the other hand, since these inks require the addition of glycerol as a viscosity agent, the evaluation of lysozyme inks was performed with or without glycerol by infrared spectroscopy Fourier transform (FTIR). An inspection of the major bands of the spectra shows that lysozyme does not change during the process (FIG. 13).

The experiments corroborated the capacity of the printing system to deliver proportional amounts of the biologic to the printed areas of the inert substrate and concentrations of the inks (Montenegro-Nicolini M., et al. (2015). The Use of Inkjet Printing for Dosing Biologics on Drug delivery Systems. In: 2015 AAPS Annual Meeting and Exposition Orlando, Fla., USA). However, the experimental studies showed that the printing process might reduce the integrity of the drug (measured as remaining activity) proportional to the amount printed. Similarly, the incorporation of an absorption enhancer had a negative effect on the integrity of the naked biologic, decreasing its activity (Montenegro-Nicolini M., et al. (2015). The Use of Inkjet Printing for Dosing biomacromolecular Actives in Drug Delivery Systems in: 42nd Annual Meeting and Exposition of the Controlled Release Society Edinburgh, Scotland).

In conclusion, these printing experiments on inert films with solutions of naked biologics allowed demonstrate the capacity of the printing system to deliver proportional amounts to the printed areas and concentrations thereof. Additionally, experimental studies of activity showed that the printing process can variably reduce the integrity of the biologic, and it was proportional to the amount printed. Similarly, the incorporation of an absorption enhancer had a negative effect on the integrity of the naked biologic, reducing its activity.

Experiments of Nanosystems Loaded with Biologics

The characterization of each nanosystem was performed by dynamic light scattering (DLS) to study size distribution and laser micro-Doppler electrophoresis (mLDE) for determining the zeta potential. Suspensions with nanosystems (also called herein nanosuspensions) were evaluated to develop the inks for printing biologics. The influence of an absorption enhancer in the nanosuspensions and its effects in printing on polymer films as printing substrates with said nanosuspension was also determined.

### Nanoemulsions and Nanocapsules

Experiments with nanoemulsions (NE) and nanocapsules (NC) were designed to find formulations useful as printing inks considering the size of the particles, uniformity, encapsulation of the biologic (using lysozyme (Lys) as a model), and post-printing activity of the biologic.

As a biologic drug example, lysozyme was used in nanoemulsions and nanocapsules as a potential buccal delivery system. The nanoemulsions and nanocapsules as lysozyme vehicles were obtained by the method of solvent displacement, where an organic phase containing oil (as an oil core of the nanostructure), a surfactant (such as lecithin, cetylpyridinium chloride, hexadecyltrimethylammonium bromide), and water-miscible organic solvents (such as acetone or ethanol), is incorporated into an aqueous solution to form a nanoemulsion (without agitation or high-energy homogenization).

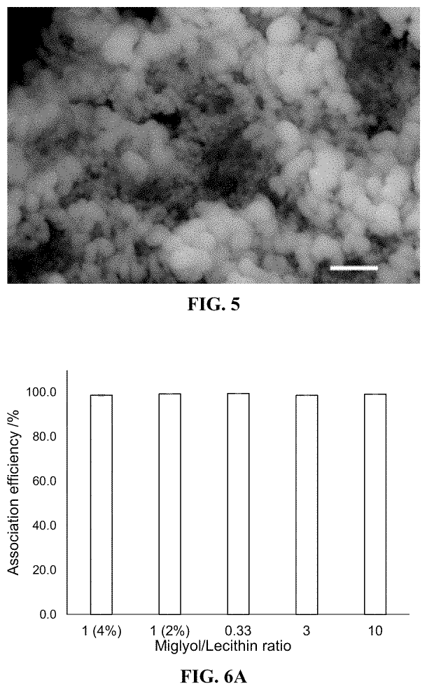

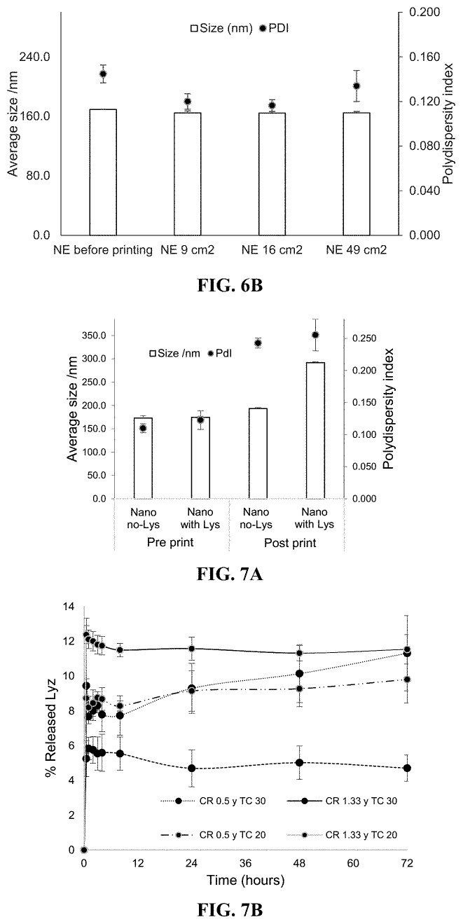

The experiment for nanoemulsions was conducted as a scan through a ternary diagram design to evaluate how the formulation results in different conditions (Table 1 and FIGS. 6A and 6B). Table 1 shows a list of the best suitable nanoemulsions among the total formulations of the ternary diagram in terms of size, polydispersity index, and zeta potential. FIG. 5 shows a SEM micrograph of an example of a nanoemulsion loaded with lysozyme.

TABLE 1

| Miglyol | Lecithin | Water | Size | | Zeta |
|---|---|---|---|---|---|
| (%) | (%) | (%) | (nm) | PDI | potential |
| 2.31 | 0.58 | 97.1 | 93.4 ± 1.0 | 0.15 ± 0.03 | −52.0 ± 2.0 |
| 10 | 10 | 80 | 280.0 ± 2.3 | 0.38 ± 0.01 | −66.7 ± 1.3 |
| 20 | 20 | 60 | 330.3 ± 6.7 | 0.39 ± 0.02 | −72.6 ± 0.8 |
| 5 | 5 | 90 | 205.3 ± 4.0 | 0.27 ± 0.02 | −79.1 ± 0.5 |
| 2 | 2 | 96 | 172.9 ± 2.9 | 0.21 ± 0.02 | −53.7 ± 1.8 |
| 1 | 1 | 98 | 244.2 ± 16 | 0.32 ± 0.02 | −54.2 ± 0.6 |
| 1 | 0.1 | 98.9 | 156.7 ± 1.7 | 0.09 ± 0.02 | −37.0 ± 0.4 |

PDI: Polydispersity index.

Table 1 shows that by increasing the oil phase (Miglyol) and surfactant (lecithin), the particle size is increased but also increases the polydispersity index (PDI). It also shows that when approaching the proportions of the reference formulation (Miglyol 2.31% and 0.58% lecithin), the size increases with a decrease in the PDI.

However, for the purposes of inkjet printing, the properties of these nanoemulsions were sufficient to allow them to be printed. FIG. 6A shows that the printing process does not affect the load of lysozyme (association efficiency and loading) in nanoemulsions formulated with different Miglyol/lecithin percentage ratios. Furthermore, FIG. 6B shows that the physicochemical characteristics (size and PDI) of the nanoemulsions loaded with lysozyme were not modified after the printing process.

Thus, printing on inert substrates with inks containing nanoemulsions loaded with lysozyme is possible, and the physicochemical characteristics of the nanoemulsion particles are maintained throughout the printing process.

The size, polydispersity index (PDI), zeta potential, and encapsulation efficiency (EE) of nanoemulsions and nanocapsules containing lysozyme formulations are shown in Table 2. Results are expressed as mean (standard deviation).

TABLE 2

| | | | | | Zeta | | | |
|---|---|---|---|---|---|---|---|---|
| | Size | | | | potential | | EE | |
| Formulation | (nm) | | PDI | | (mV) | | (%) | |
| NE + Lys | 169.2 | (0.4) | 0.14 | (0.01) | −12.8 | (0.4) | 81.4 | (0.4) |
| 1% | | | | | | | | |
| NC EPO | 209.8 | (0.9) | 0.20 | (0.01) | 83.2 | (0.5) | 87.0 | (2.4) |
| 0.1% + | | | | | | | | |
| Lys 1% | | | | | | | | |
| NC EPO | 193.7 | (3.3) | 0.14 | (0.01) | 84.4 | (2.3) | 109.9 | (5.0) |
| 0.05% + | | | | | | | | |
| Lys 1% | | | | | | | | |
| NE + Lys | 200.5 | (1.4) | 0.13 | (0.02) | −38.5 | (1.2) | 39.5 | (1.3) |
| 10% | | | | | | | | |
| NC EPO | 226.3 | (1.6) | 0.20 | (0.01) | 76.1 | (1.7) | 18.0 | (0.8) |
| 0.1% + | | | | | | | | |
| Lys 10% | | | | | | | | |
| NC EPO | 187.9 | (0.7) | 0.27 | (0.01) | 83.4 | (0.3) | 17.8 | (0.1) |
| 0.05% + | | | | | | | | |
| Lys 10% | | | | | | | | |

Table 2 shows the zeta potential inversion from the nanoemulsion (expressing the negatively charged surfactant) to Eudragit® EPO nanocapsules (polycation). Better encapsulation efficiency was observed at low lysozyme loads, indicating that the emulsification system has a maximum incorporation capacity.

### Protein-Coated Nanoparticles

The manufacture of protein-coated nanoparticles by antisolvent co-precipitation can be developed by varying the core co-precipitation molecule. FIG. 11A shows the effect of different core precursors (glycine, D,L-Valine, K₂SO₄, NaCl, and lactose) on the size and polydispersity index of the nanoparticles. FIG. 11B shows a SEM micrograph of an example of nanoparticles D,L-valine typically obtained by antisolvent co-precipitation.

The effect of the co-precipitant type (core precursor) on the average size and polydispersity index at different surfactant concentrations (SMS) is shown in Table 3. That table shows the results obtained using various concentrations of sorbitan monostearate (SMS, as surfactant) with saccharides such as lactose, mannitol, and sorbitol. In terms of the size and polydispersity (PDI) of these nanoparticles, a control in size and dispersion was observed for lactose and mannitol formulations, whereas the use of sorbitol only allowed a tighter control at low SMS concentrations (Le, A. -D., et al. (2014). Core Forming Antisolvent Coprecipitation of Protein Crystals Loaded. In: 2014 AAPS Annual Meeting and Exposition. San Diego, Calif., USA).

TABLE 3

| | Lactose | | Mannitol | | Sorbitol | |
|---|---|---|---|---|---|---|
| SMS concentration | Size | | Size | | Size | |
| (μM) | (nm) | PDI | (nm) | PDI | (nm) | PDI |
| 0 | 150.1 | 0.061 | 184.1 | 0.245 | 185.7 | 0.162 |
| 8 | 151.9 | 0.170 | 185.4 | 0.248 | 165.3 | 0.218 |
| 15 | 138.7 | 0.080 | 186.0 | 0.094 | 519.1 | 0.735 |
| 30 | 146.1 | 0.175 | 175.0 | 0.138 | 488.2 | 0.814 |
| 45 | 134.1 | 0.220 | 185.6 | 0.279 | 533.9 | 0.404 |
| 60 | 149.8 | 0.102 | 159.6 | 0.150 | 162.5 | 0.322 |

The results indicate that only some core materials are compatible with the process of co-precipitation by antisolvent (such as the aforementioned saccharides, i.e., lactose, mannitol, and sorbitol) according to the properties of average size and zeta potential (Le, A. -D., et al. (2014). Co-precipitation Antisolvent Synthesis of D,L-Valine/Lysozyme. In: 2014 AAPS Annual Meeting and Exposition. San Diego, Calif., USA).

### Polymeric Nanoparticles

For the development of polymeric nanoparticles, a complex coacervation technique was used, and it follows the electrostatic interaction of a polycation (a polymer with cationic monomer units) with a polyanion (a polymer with anionic monomeric units). Moreover, along a study of charge ratio (positive charges/negative charges, n⁺/n⁻), the formation of nanoparticles was observed only in the presence of an excess of polyanion.

The process of printing on an inert substrate using a suspension with a nanosystem as carriers of biologic drug models was performed using polymeric nanoparticles. The suspension was incorporated into a cartridge for inkjet printing. The particle properties were measured before and after the printing process. Table 4 shows the effect of the printing process on polymeric nanoparticles (NPs) in a model of ink. Results are expressed as means (standard deviation). Except for an increase in the polydispersity index (PDI), the particles maintain their properties during the printing process and define the methodology for incorporating drug loaded nanoparticles on polymer films.

TABLE 4

| | Size | Zeta potential | Polydispersity |
|---|---|---|---|
| Test condition | (nm) | (mV) | index |
| NPs before printing | 214.3 (1.3) | −34.1 (0.3)* | 0.17 (0.02)** |
| NPs after printing | 226.0 (17.8) | −26.0 (3.8)* | 0.43 (0.11)** |

*, **differences observed are statistically significant (p < 0.05);
NPs: nanoparticles

The following experiments with a synthetic polycation, a polymethacrylate derivative with ionizable tertiary amino groups in its monomeric units (Eudragit® E PO) and alginate, resulted in polymeric nanoparticles with excellent size, polydispersity index, and zeta potential.

### Examples of Nanosuspensions as Printing Inks

The polymer nanoparticles loaded with lysozyme were obtained by mixing controlled volumes of a solution with Eudragit® E (EPO) (a cationic derivative of polymethacrylate) at a concentration of 2.5 mg/mL and a solution with alginate (anionic polymer) at a concentration of 2.5 mg/mL. The solutions were mixed under constant magnetic stirring (300 rpm) at room temperature. The different formulations are shown in Table 5.

TABLE 5

| | | | TC | | | | | |
|---|---|---|---|---|---|---|---|---|
| n⁺ | n⁻ | | (n⁺ + n⁻) | Conc | Conc | Vol | Vol | |
| μmol | μmol | CR | μmol | EPO | Alg | EPO | Alg | Size ± SD |
| charges | charges | (n⁺/n⁻) | charges | μg/μL | μg/μL | μL | μL | nm |
| 27.3 | 2.7 | 10 | 30 | 2.5 | 2.5 | 3,032.7 | 216.1 | 140.7 ± 2.2 |
| 24 | 6 | 4 | 30 | 2.5 | 2.5 | 2,668.8 | 475.5 | 132.4 ± 3.7 |
| 20 | 10 | 2 | 30 | 2.5 | 2.5 | 2,224.0 | 792.4 | 143.2 ± 6.1 |
| 10 | 20 | 0.5 | 30 | 2.5 | 2.5 | 1,112.0 | 1,584.9 | 236.4 ± 4.0 |
| 6 | 24 | 0.25 | 30 | 2.5 | 2.5 | 667.2 | 1,901.9 | 212.7 ± 5.0 |
| 2.7 | 27.3 | 0.1 | 30 | 2.5 | 2.5 | 303.3 | 2,161.2 | 191.2 ± 13.2 |

n⁺: Positive charges;
n⁻: Negative charges;
CT (n⁺/n⁻): Charge ratio;
TC (n⁺ + n⁻): Total charge sum;
Conc EPO: Eudragit ® E concentration;
Conc Alg: Alginate concentration;
Vol EPO: Volume of Eudragit ® E solution;
Vol Alg: Volume of alginate solution;
SD: Standard deviation.

Based on the polymer mass of the mixture reaction, 10% w/w lysozyme was included in each formulation from a stock solution of 1 mg/mL of lysozyme. 1.5 mL of each resulting formulation was centrifuged at 13000 rpm for 30 minutes, and 50 μL of glycerol was added for redispersing. After removing the supernatant, the pellet was resuspended with an aqueous solution with 30% v/v of glycerol (as ink viscosity agent) for obtaining a suspension. The suspensions obtained were used as printing inks. An absorption enhancer, such as deoxycholate, was added to the suspensions in some experiments.

Similarly, polymeric nanoparticles loaded with insulin were prepared by mixing controlled volumes of solutions with Eudragit E (EPO) at a concentration of 2.5 mg/mL and solutions with alginate at a concentration of 2.5 mg/mL. The solutions were mixed under constant magnetic stirring (300 rpm) at room temperature. The different formulations are shown in Table 6.

TABLE 6

| | | | TC | | | | | |
|---|---|---|---|---|---|---|---|---|
| n⁺ | n⁻ | | (n⁺ + n⁻) | Conc | Conc | Vol | Vol | |
| μmol | μmol | CR | μmol | EPO | Alg | EPO | Alg | Size ± SD |
| charges | charges | (n⁺/n⁻) | charges | μg/μL | μg/μL | μL | μL | nm |
| 20 | 10 | 2 | 30 | 2.5 | 2.5 | 2224.0 | 792.4 | 191.9 ± 4.5 |
| 10 | 20 | 0.5 | 30 | 2.5 | 2.5 | 1112.0 | 1584.9 | 182.4 ± 1.7 |

n⁺: Positive charges;
n⁻: Negative charges;
CT (n⁺/n⁻): Charge ratio;
TC (n⁺ + n⁻): Total charge sum;
Conc EPO: Eudragit ® E concentration;
Conc Alg: Alginate concentration;
Vol EPO: Volume of Eudragit ® E solution;
Vol Alg: Volume of alginate solution;
SD: Standard deviation.

Based on the polymer mass of the mixture reaction, 10% w/w insulin was included in each formulation from a stock solution of 1 mg/mL of insulin. 1.5 mL of each resulting formulation was centrifuged at 13000 rpm for 30 minutes, and 50 μL of glycerol was added for redispersing. After removing the supernatant, the pellet was resuspended with an aqueous solution with 30% v/v of glycerol (as ink viscosity agent) for obtaining a suspension. The suspensions obtained were used as printing inks. An absorption enhancer, such as deoxycholate, was added to the suspensions in some experiments.

The obtained printing inks with lysozyme or insulin were loaded into Hewlett Packard cartridges compatible with Hewlett Packard Deskjet 1000 printer, and it was installed to run the printing experiments. Using a commercial software tool appropriate for the task, surfaces of 3×3 cm² were printed on an inert substrate to evaluate the effect of the printing process on the polymeric nanoparticles. After the printing process, the printed polymeric nanoparticles were recovered by refluxing with 1 mL of Milli-Q® water, and their physicochemical properties in terms of size, polydispersity index, and zeta potential were determined.

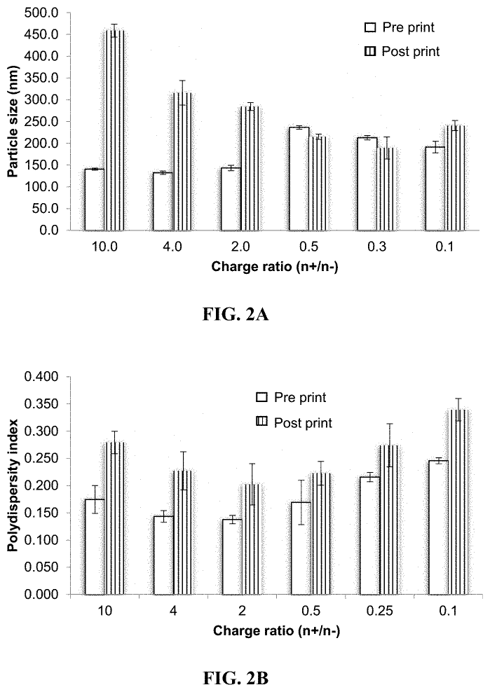

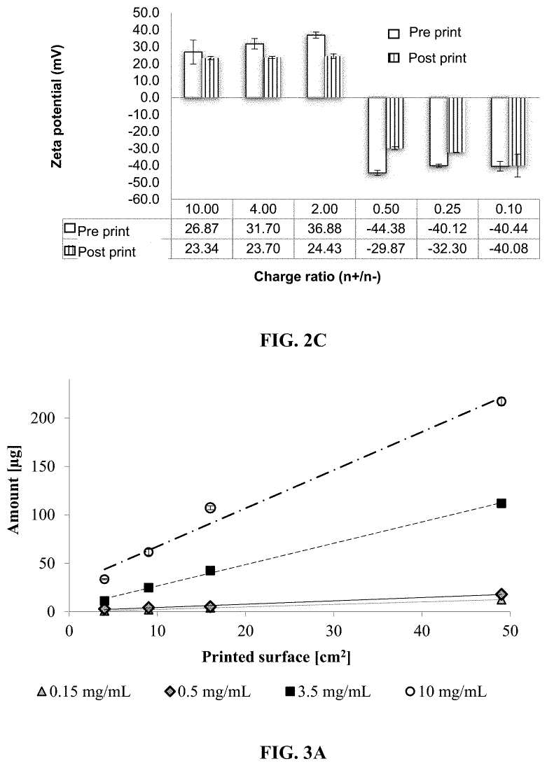

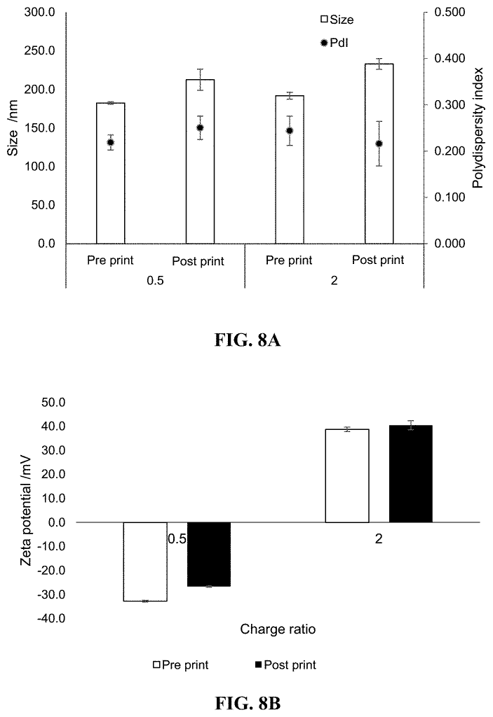

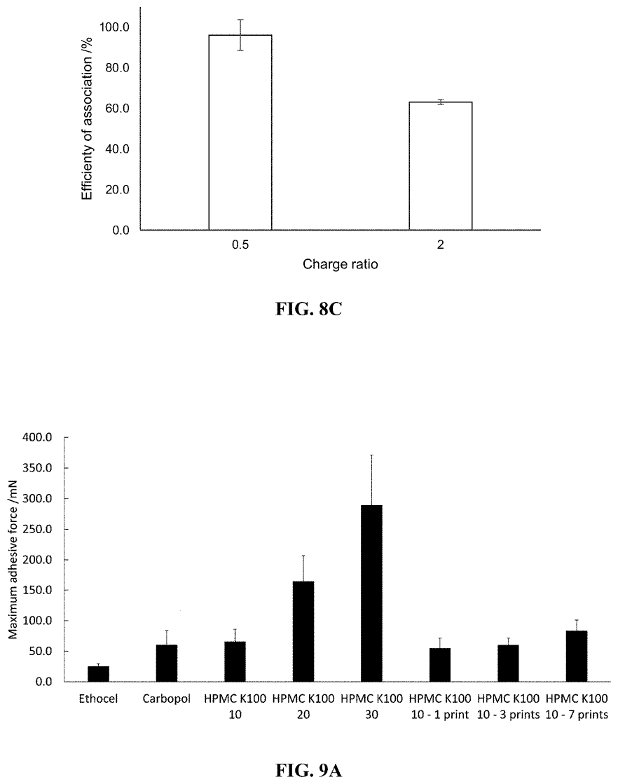

The results of the printing process effects on the polymeric nanoparticles loaded with lysozyme contained in the printing ink are shown in FIGS. 2A, 2B, and 2C. Except for an increase in the polydispersity index, the particles containing lysozyme as a model maintain their properties during the printing process and define the methodology for incorporating drug loaded nanoparticles on polymer films. The results for the polymeric nanoparticles loaded with insulin are shown in FIGS. 8A and 8B. A slight increase in particle size was observed for insulin-loaded polymeric nanoparticles but always maintained suitable polydispersity and size characteristics in the nanometer range.

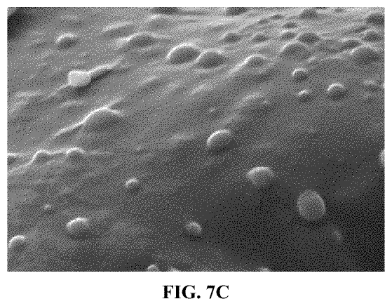

As shown in FIG. 7A, the printing process does not alter the physicochemical characteristics of polymeric nanoparticles obtained by coacervation of Eudragit® E and alginate at different charge ratios (CR) and different total charges (TC) on the number of positive and negative charges, which provide each polymer forming the nanoparticle. As shown in FIG. 7B, a controlled and reduced release can always be maintained in the first 72 hours, regardless of the CR and TC conditions and after subjecting the particles to the printing process. This profile ensures that such nanocarriers can carry the drug (biologic) protected from environmental conditions (and the printing process) to the therapeutic target where it can be released. FIG. 7C shows the typical appearance of these spherical polymeric nanoparticles.

Experimental studies of polycation and polyanion (n⁺/n⁻) and the sum of total charges provided to the reaction (in Table 7, n⁺+n⁻=6 μM) to determine their influence on the characteristics of the nanoparticles show a tendency to lower the average sizes as the charge ratio increases, relatively independent of the total charge amount. Smaller sizes become apparent from a charge ratio of 0.5 (FIG. 8A), which coincides with that observed in the zeta potential, where the ratio of 0.5 is the inversion of negative (predominance of polyanion) to positive charges (predominance of polycation) (FIG. 8B) occurs. Moreover, it was found that the ratio of 0.5 led to a higher association efficiency in comparison with the charge ratio of 2 (FIG. 8C), indicating that insulin has a stronger affinity to the nanoparticles when a net negative charge predominates.

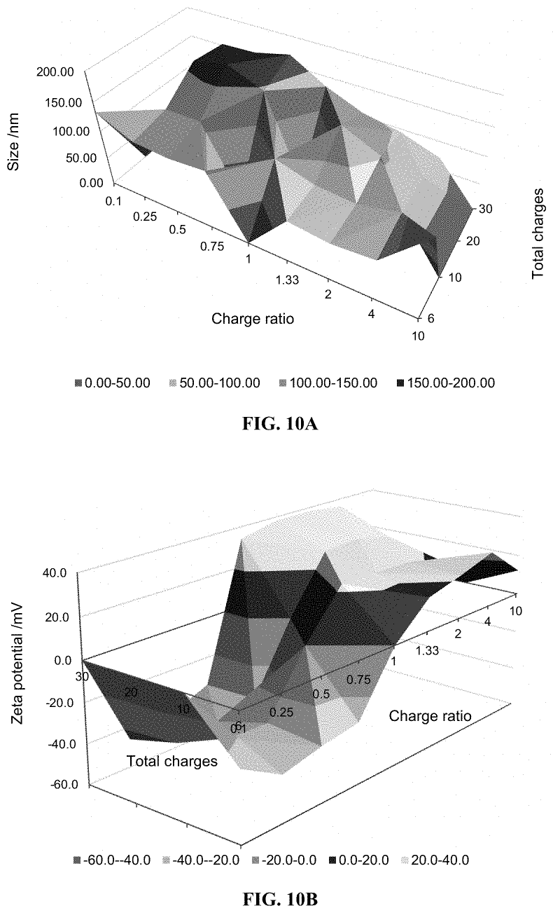

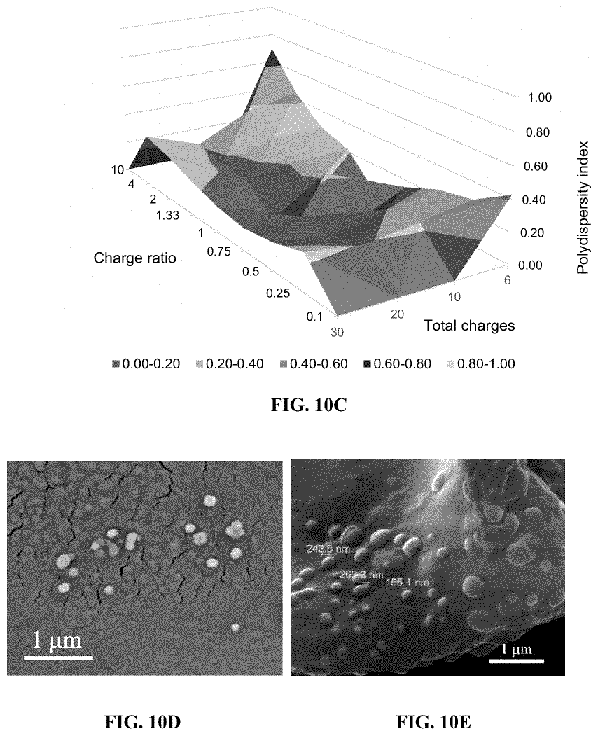

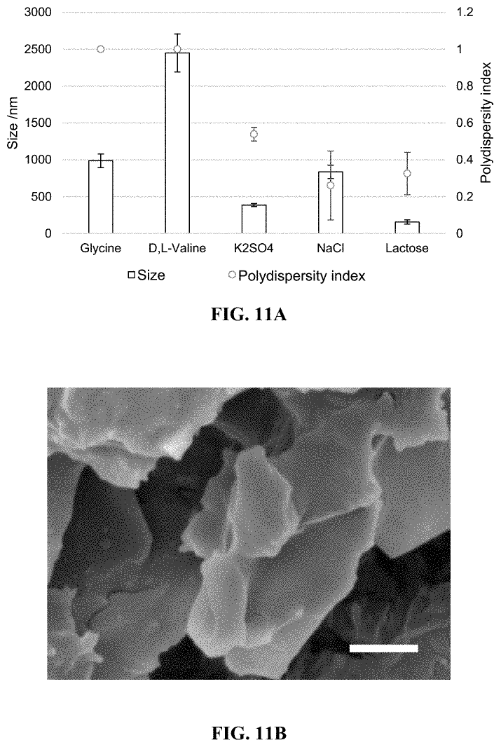

However, the results of the polydispersity index indicate that the region between the charge ratio of 0.25 to 0.75 is the one combining smaller sizes, stronger zeta potential, and lower polydispersity indices (FIG. 10A, 10B, and 10C). Polymeric nanoparticles exhibit spherical morphology by scanning electron microscopy (SEM), and their size determined by dynamic light scattering (DLS) correlates to that of SEM (FIGS. 10D and 10E).

TABLE 7

| Charge ratio | Size | | Polydispersity | | Zeta potential | |
|---|---|---|---|---|---|---|
| (n⁺/n⁻) | (nm) | | index | | (mV) | |
| 0.1 | 123.8 | (4.9) | 0.213 | (0.015) | −33.2 | (2.4) |
| 0.25 | 137.6 | (2.6) | 0.112 | (0.001) | −33.2 | (0.6) |
| 0.5 | 119.5 | (2.0) | 0.149 | (0.009) | 40.0 | (1.2) |
| 0.75 | 76.2 | (3.3) | 0.291 | (0.034) | 37.8 | (2.6) |
| 1 | 56.8 | (1.0) | 0.283 | (0.023) | 36.1 | (3.0) |
| 1.33 | 59.1 | (0.8) | 0.216 | (0.007) | 27.5 | (36.6) |
| 2 | 57.8 | (7.0) | 0.330 | (0.075) | 62.6 | (32.7) |
| 4 | 66.2 | (3.5) | 0.366 | (0.012) | 87.6 | (39.5) |
| 10 | 92.9 | (19.5) | 0.639 | (0.046) | 30.9 | (14.7) |

Table 7 shows the effect of the charge ratio between Eudragit® E PO (polycation) and alginate (polyanion) on the properties of size, polydispersity, and zeta potential of polymeric nanoparticles. This table corresponds to the total sum of charges (n⁺+n⁻) equal to 6 μM in experiments exploring the range comprised of 2, 4.4, 6, 10, and 20 μM. Results are expressed as mean (standard deviation).

### Examples of Polymeric Films

To evaluate the effect of model printing routines on polymer films for buccal drug delivery, hydroxypropylmethyl cellulose (HPMC) films of different grades were manufactured by solvent casting technique.

Aqueous solutions of HPMC K3, K100, and K15M 10% w/v were prepared, and over a solid polymer base, films were incorporated with 10% w/w glycerol as a plasticizer in the casting solution. Constant magnetic stirring (500 rpm) at room temperature was used to dissolve until a translucent solution. After the complete dissolution of the polymer, these solutions were stored at 4 ° C. for 24 hours to remove air bubbles from the solution. Subsequently, 10, 20, or 30 g of these solutions were cast in polytetrafluoroethylene molds (PTFE or Teflon) and left to dry in an air current until constant weight for at least 24 hours. After drying, the polymer films were demolded and used in the printing process.

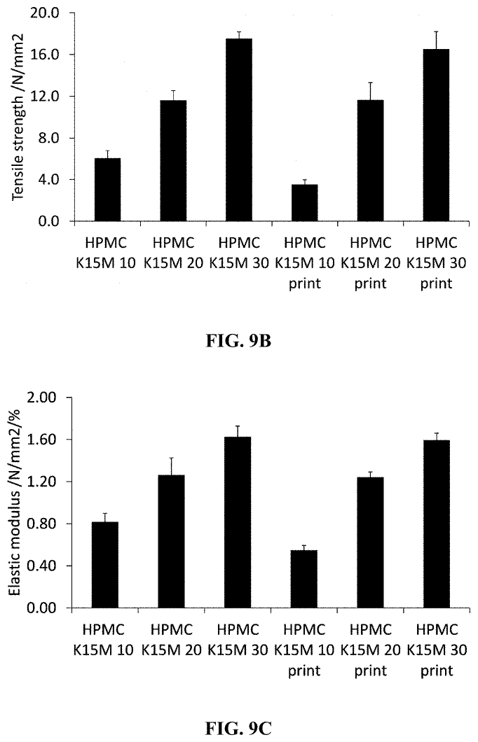

As model inks, aqueous solutions of glycerol 30% v/v were used, and the effect that the process of printing on films in terms of mechanical properties such as elongation at break, tensile strength, and elastic modulus was evaluated (FIGS. 9B and 9C). Due to the potential of these films as dosage forms for buccal administration, their pre and post-printing mucoadhesive properties were evaluated and compared to Carbopol and ethyl cellulose, other polymers with different mucoadhesive properties and conventionally used (FIG. 9A).

These experiments with various types of hydroxypropylmethyl cellulose (HPMC K3, K100, and K15M) showed that after the printing process, the mechanical and morphological properties of the polymer films do not vary (Alvarez R., et al. (2014). Study of polymeric films as substrates for inkjet printing of drug delivery systems. In: AAPS National Biotechnology Conference 2014 San Diego, Calif., USA). The mucoadhesive properties are diminished but do not prevent the use of the film as a dosage form.

### CONCLUSIONS

The above experiments provided results for the preparation process of the printing inks as well as the preparation process of the polymeric nanoparticles by coacervation or nanoemulsions with Miglyol core. These printing inks had particles with a size range between 100 and 300 nm, depending on their nature, and a wide range of biologic encapsulation efficiencies (50 to 95% of association). These studies showed that release kinetics controls the release of lysozyme (one biologic model), and also depends on the nature of the nanosystem particle.

One of the most remarkable and illustrating operations of the present invention data is shown in FIGS. 2A, 2B, and 2C. This corresponds to a formulation example before and after being subjected to the printing process and shows that the particle characteristics change little during the process. Load and protein activity studies of a biologic model (lysozyme) indicate that the amount immobilized at the particle, together with the biologic structure, are preserved during the printing process. Another notable example is seen in FIGS. 8A and 8B, which detail the variation in size and polydispersity of two formulations of polymeric nanoparticles containing insulin. It is noted that the size increases in about 50 nm after the printing process but always results in formulations with nanoparticles averaging sizes less than 250 nm.

What is claimed is:

1. Method for the manufacture of a dosage form with mucoadhesive properties for buccal administration of a biologic comprising:
a) preparing polymeric nanoparticles including at least one biologic, wherein the polymeric nanoparticles are prepared by mixing a polymer with cationic monomeric units and a polymer with anionic monomeric units in a charge ratio (n+/n−) of 0.1 to 1;
b) preparing a printing ink with a suspension of the polymeric nanoparticles including the at least one biologic; and
c) printing onto a polymeric film with said printing ink;
wherein the biologic is selected from a lymphokine, a hormone, hematopoietic factors, growth factors, antibodies, enzymes, inhibitors, and vaccines;
wherein said lymphokine is selected from aldesleukin cytokine, antineoplastic protein denileukin diftitox, recombinant interleukin Oprelvekin, interferon-α1, interferon α2a, interferon-α2b, interferon β1a, interferon β1b, interferon γ1b, and tumoral necrosis factor human-α1a (TNFα-1a) tasonermin;
wherein said hormone is selected from human insulin, insulin lispro, insulin aspart, insulin glulisine, insulin glargine, insulin detemir, glucagon, somatropin, somatrem, follitropin-α, follitropin-β, choriogonadotropin-α, lutropin-α, calcitonin, teriparatide, preotact, thyrotropin-α, nesiritide, and angiotensin 1-9;
wherein said hematopoietic factors are selected filgrastim, lenogastrim, sargramostim, molgramostim, epoetin-α, epoetin-β, epoetin-γ, and darbepoetin-α;
wherein said growth factors are selected from mecasermin, mecasermin rinfabate, nepidermin, becaplermin, palifermin, dibotermin-α, and eptotermin-α;
wherein said antibodies are selected from Fab fragments such as arcitumomab, digoxin Fab, abciximab, certolizumab; murine antibodies such as muramonab-CD3, capromab, ibritumomab tiuxetan, toistumomab; chimeric antibodies such as rituximab, infliximab, basiliximab, cetuximab, brentuximab vedotin; humanized antibodies such as daclizumab, trastuzumab, palivizumab, gemtuzumab ozogamicin, alemtuzumab, efalizumab, omalizumab, bevacizumab, natalizumab, ranbizumab, eculizumab, tocilizumab; human antibodies such as adalimumab, panitumumab, golimumab, canakinumab, ustekinumab, ofatumumab, denosumab, belimumab, and ipilimumab;
wherein said enzymes are selected from imiglucerase, agalsidase-β, alglucosidase-α, laronidase, idursulfase, galsulfase, factor VIIa, factor VIII, factor IX, drotrecogin-α, alteplase, reteplase, tenecteplase, dornase-α, rasburicase, lysozyme, and ribonuclease;
where such inhibitors are selected from desirudin, lepirudin, antithrombin III, ecallantide, and anakinra; and
where such vaccines are selected from human hepatitis vaccine and human papilloma virus vaccine.

2. The method of claim 1, wherein the biologic is selected from the group consisting of lysozyme, ribonuclease, angiotensin 1-9, and insulin.

3. The method of claim 1, wherein the polymer with cationic monomeric units is a polymethacrylate derivative with ionizable tertiary amine groups in its monomeric units.

4. The method of claim 1, wherein the polymer with anionic monomeric units is alginate.

5. The method of claim 1, wherein for preparing the printing ink, a viscosity agent is added to the suspension of the polymeric nanoparticles, said viscosity agent is selected from the group consisting of glycerol and propylene glycol.

6. The method of claim 5, wherein the viscosity agent is glycerol.

7. The method of claim 1, wherein for preparing the printing ink, an absorption enhancing agent is added to the suspension of the polymeric nanoparticles.

8. The method of claim 7, wherein the absorption enhancing agent is selected from deoxycholic acid, taurocholic acid, glycodeoxycholic acid, glycocholic acid, and taurodeoxycholic acid.

9. The method of claim 1, further comprising preparing the polymeric film with a polymer selected from the group consisting of cellulose, cellulose derivatives, polyvinyl pyrrolidone, polyvinyl alcohol, chitosan, alginate, agar, carrageenan, guar gum, xanthan gum, polycarbophil, polyacrylic acid derivatives, poly(methacrylic acid) derivatives, and combinations thereof.

10. The method of claim 9, wherein the poly(methacrylic acid) derivative is a cationic polymethacrylate.

11. The method of claim 9, wherein the cellulose derivative is selected from the group consisting of hydroxypropyl cellulose, hydroxyethyl cellulose, and carboxymethyl cellulose.

12. The method of claim 9, wherein for preparing the polymeric film, an aqueous solution of the polymer is used in a concentration of 10% w/v.

13. The method of claim 12, wherein a plasticizing agent is added to the aqueous solution of the polymer for obtaining a ratio range of plasticizing agent:polymer of 1:9 to 3:7 in the polymeric film.

14. The method of claim 13, wherein the plasticizing agent is glycerol.
